(19) United States
(12) Patent Application Publication
(10) Pub. No.: US 2022/0091815 A1
(43) Pub. Date: Mar. 24, 2022

(54) SYSTEMS AND METHODS TO FACILITATE REAL-TIME EDITING BY VOICE BASED ON INDIVIDUAL INPUTS FROM A USER AND TIMING INFORMATION FOR THE INDIVIDUAL INPUTS

(71) Applicant: Suki AI, Inc., Redwood City, CA (US)

(72) Inventors: Sudheer Tumu, Santa Clara, CA (US); Yashas Rao, San Fransisco, CA (US); Maneesh Dewan, Sunnyvale, CA (US); Arunan Rabindran, San Mateo, CA (US); Nithyanand Kota, Santa Clara, CA (US); Jatin Chhugani, Mountain View, CA (US)

(21) Appl. No.: 16/948,494

(22) Filed: Sep. 21, 2020

(51) Int. Cl. G06F 3/16; G10L 15/22; G10L 15/30; G06F 3/0481

(52) U.S. Cl. CPC G06F 3/167; G10L 15/22; G10L 2015/223; G06F 3/0481; G10L 15/30

(57) ABSTRACT

Systems and methods to facilitate real-time editing by voice based on voice and physical inputs from a user and timing information for the individual inputs are disclosed. Exemplary implementations may: generate output signals conveying physical manipulation of a physical user interface by a user; generate output signals conveying audio information; process the output signals of the physical user interface to generate a physical input stream; process the captured audio information to generate an audio stream that represents spoken inputs uttered by the user; synchronize the physical input stream and the audio stream to convey relative timing between inputs both in the form of manipulations and in the form of spoken inputs included in the captured audio information; store the synchronized physical input stream and audio stream; determine, from the synchronization, sets of inputs that correspond to different individual commands; and execute the commands corresponding to the sets of inputs.

### FIELD OF THE DISCLOSURE

The present disclosure relates to facilitating real-time editing by voice based on individual inputs from a user and timing information for the individual inputs.

### BACKGROUND

Real-time editing using voice may involve an interaction (e.g., by voice, by tapping, by keyboard, by mouse) indicating where in a document to edit followed by speech from a user characterizing words and/or phrases to replace or insert into the document. Current techniques for divining user intent may not provide accurate and/or reliable results.

### SUMMARY

One aspect of the present disclosure relates to determining user inputs to a client computing platform through spoken phrases and manual inputs. The client computing platform may contemporaneously capture an audio stream and a physical input stream that represent spoken inputs and manual inputs, respectively, of the user to the client computing platform. The audio stream may define the spoken inputs by a user characterizing words and/or phrases that comprise commands related to a document. Individual portions of, or moments in, the audio stream (e.g., for individual words and/or phrases) may be associated with a timestamp. The interaction stream may capture individual physical inputs generated by the user via a physical user interface of a client computing platform (e.g., touchscreen of a smartphone), and/or other physical user interface(s). Some of the physical inputs captured may specify locations of the document to edit based on individual interactions (e.g., a tap via the client computing platform), actions to be performed to indicated text or images, and/or other actions. The individual physical inputs may be associated with a timestamp. The audio stream and the physical input stream may be synchronized based on time. The capture may be performed at the client computing platform. The synchronization of the audio stream with the physical input stream may be performed at a server. Such synchronization may facilitate determining the command to process and execute based on the timestamps of both the physical inputs and the spoken inputs.

One aspect of the present disclosure relates to a system configured to facilitate real-time editing by voice based on individual inputs from a user and timing information for the individual inputs. The system may include one or more client computing platforms, one or more hardware processors configured by machine-readable instructions, and/or other components. Machine-readable instructions may include one or more instruction components. The instruction components may include computer program components. The instruction components may include one or more of an information processing component, a stream synchronization component, a command processing component, and/or other instruction components.

Individual physical user interfaces may be configured to generate output signals conveying physical manipulation of the physical user interface by a user. The individual physical user interfaces may be associated with individual client computing platforms. The user may be enabled to generate physical inputs to the client computing platform through the physical manipulation of the physical user interface. Individual inputs may be associated with commands for the client computing platform.

Individual audio input sensors may be configured to generate output signals conveying audio information. The individual audio input sensor may be associated with the individual client computing platforms. The audio information may define audio content captured by the audio input sensor. The audio content may include spoken inputs uttered by the user. The inputs may include the physical inputs and the spoken inputs.

The information processing component may be configured to process the output signals of the physical user interface to generate a physical input stream. The physical input stream may represent the individual physical inputs of the user to the physical user interface. The physical input stream may convey the individual physical inputs by the user through the physical interface, timing information for the individual physical inputs, and/or other information.

The information processing component may be configured to process the captured audio information to generate an audio stream. The audio stream may represent the spoken inputs uttered by the user, timing information of the spoken inputs, and/or other information.

The stream synchronization component may be configured to synchronize the physical input stream and the audio stream to convey relative timing between inputs to the client computing platform. The relative timing may include inputs to the client computing platform both (i) in the form of manipulations of the physical user interface and (ii) in the form of spoken inputs included in the captured audio information. The stream synchronization component may be configured to store the synchronized physical input stream and audio stream.

The command processing component may be configured to determine, from the synchronized physical input stream and audio stream, sets of inputs that correspond to different individual commands. As such, a first set of inputs may be determined corresponding to a first command, and a second set of inputs is determined corresponding to a second command. The second set of inputs may be separate and discrete from the first set of inputs. The first set of inputs may include a first input in the form of a manipulation of the physical user interface and a second input that is a spoken input. The second set of inputs may include a third input in the form of a manipulation of the physical user interface and fourth input that is a spoken input.

The command processing component may be configured to execute the commands corresponding to the sets of inputs such that the first command and the second command may be executed.

As used herein, the term “obtain” (and derivatives thereof) may include active and/or passive retrieval, determination, derivation, transfer, upload, download, submission, and/or exchange of information, and/or any combination thereof. As used herein, the term “effectuate” (and derivatives thereof) may include active and/or passive causation of any effect, both local and remote. As used herein, the term “determine” (and derivatives thereof) may include measure, calculate, compute, estimate, approximate, generate, and/or otherwise derive, and/or any combination thereof.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTION OF THE DRAWINGS

FIG. 1 illustrates a system configured to facilitate real-time editing by voice based on individual inputs from a user and timing information for the individual inputs, in accordance with one or more implementations.

FIG. 2 illustrates a portion of the system configured to facilitate real-time editing by voice based on individual inputs from a user and timing information for the individual inputs, in accordance with one or more implementations.

FIG. 3A-B illustrates an example implementation of the system configured to facilitate real-time editing by voice based on individual inputs from a user and timing information for the individual inputs, in accordance with one or more implementations.

FIG. 4 illustrates an example implementation of the system configured to facilitate real-time editing by voice based on individual inputs from a user and timing information for the individual inputs, in accordance with one or more implementations.

FIG. 5 illustrates an example implementation of the system configured to facilitate real-time editing by voice based on individual inputs from a user and timing information for the individual inputs, in accordance with one or more implementations.

FIG. 6 illustrates a method to facilitate real-time editing by voice based on individual inputs from a user and timing information for the individual inputs, in accordance with one or more implementations.

### DETAILED DESCRIPTION

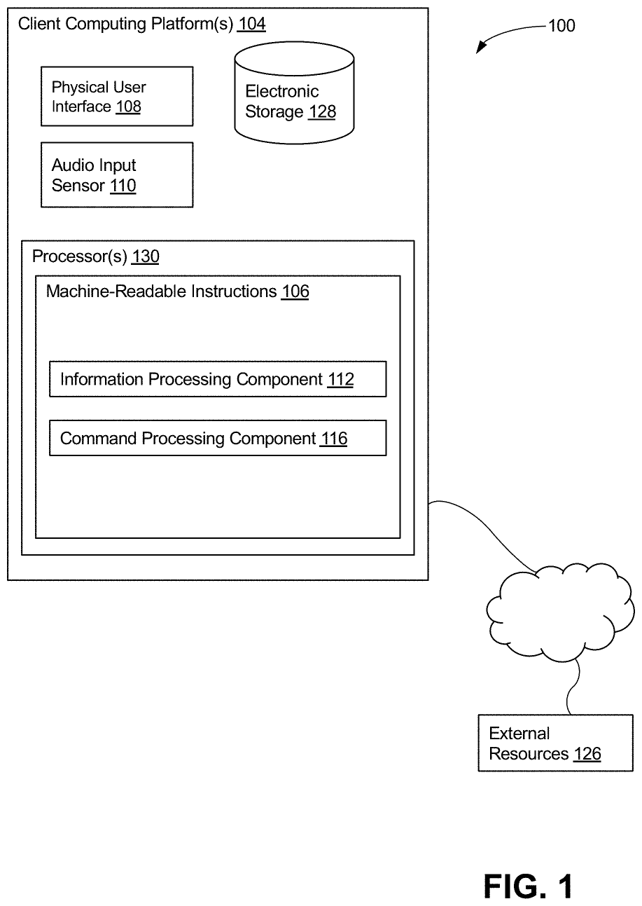

FIG. 1 illustrates a system 100 configured to facilitate real-time editing by voice based on individual inputs from a user and timing information for the individual inputs, in accordance with one or more implementations. In some implementations, system 100 may include one or more servers 102, one or more client computing platform 104, and/or other components. Server(s) 102 may be configured to communicate with one or more client computing platforms 104 according to a client/server architecture and/or other architectures. Client computing platform(s) 104 may be configured to communicate with other client computing platforms via server(s) 102 and/or according to a peer-to-peer architecture and/or other architectures. Users may access system 100 via client computing platform(s) 104.

Client computing platform(s) 104 include physical user interface 108, audio input sensor 110, processor(s) 130, and/or other components. Client computing platform(s) 104 may be configured by machine-readable instructions 106. Machine-readable instructions 106 may include one or more instruction components. The instruction components may include computer program components. The instruction components may include one or more of information processing component 112, command processing component 116, and/or other instruction components.

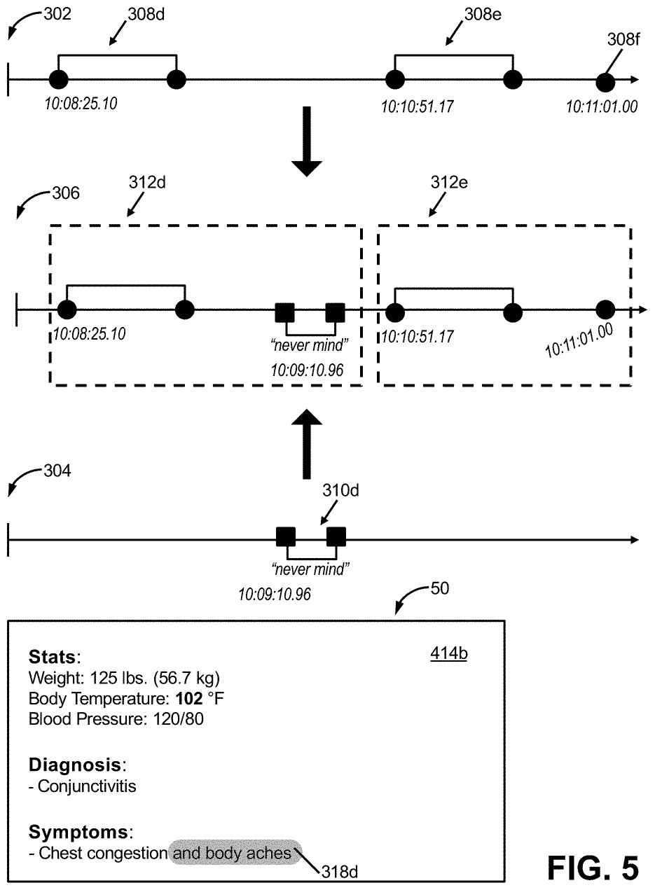

Physical user interface 108 may be configured to generate output signals conveying physical manipulation of the physical user interface by a user. Physical user interface 108 may be associated with client computing platform 104. Physical user interface 108 may include one or more of a keyboard, a mouse, a trackpad, a touchscreen, a button, a keypad, a controller, a trackball, a joystick, a stylus, and/or other physical user interfaces. By way of non-limiting example, the user may be a doctor, healthcare personnel, a scribe, a clerk, a student, and/or other users. The user may be enabled to generate physical inputs to client computing platform 104 through the physical manipulation of physical user interface 108. Physical manipulation of the physical user interface 108 to generate the physical inputs may include a screen tap of the touchscreen, a screen drag of a part of the touchscreen, a touch-and-hold of a part of the touchscreen, clicking of the mouse, pressing of the buttons, keystrokes of the keyboard, movement of the trackball (e.g., to move a cursor), utilization of the stylus on the touchscreen, and/or other physical manipulation. The physical inputs may be defined by the physical manipulations and physically communicate commands or at least portions thereof. Individual inputs may be associated with commands for client computing platform 104. In some implementations, the commands may include editing a text presented via a display of client computing platform 104, emphasizing the text (e.g., highlighting, bolding, underlining, italicizing, etc.), moving the text, copying the text, editing a name of a file, flagging the file, moving the file, copying the file, deleting the file, and/or other commands. In some implementations, particular physical inputs may specify termination of an instigated command. For example, clicking a particular button may terminate or abort a command that has been started by a previous physical input (e.g., See, FIG. 5). The inputs may include the physical inputs and spoken inputs.

Audio input sensor 110 may be configured to generate output signals conveying audio information and/or other information. Audio input sensor 110 may be associated with the client computing platform 104. The audio input sensor 110 may include a microphone and/or other audio components. The audio information may define audio content captured by the audio input sensor and/or other information. The audio content may include the spoken inputs uttered by the user. The spoken inputs may include utterances of the commands, or at least portions thereof, by the user to communicate the commands. As such, the audio stream may include one or more spoken words by the user, spoken phrases by the user, and/or other spoken inputs by the user. The spoken phrases may include multiple spoken words. For example, the spoken phrases may include “update to 120”, “insert that patient has a chest congestion”, and/or “highlight”. In some implementations, particular spoken words and/or spoken phrases may be determined as relevant to a particular command or indicate the particular command. In some implementations, particular words and/or phrases to be spoken by the user may be pre-set (e.g., by an administrator, a healthcare system, the user) as relevant to the particular command. In some implementations, particular spoken inputs may be determined as relevant or be relevant to the termination or abortion of a command already instigated by a physical input. For example, “never mind” may be a spoken phrase that is a spoken input that may abort an instigation of a command (e.g., See, FIG. 5).

Upon audio input sensor 108 generating the output signals to convey the audio information, information processing component 112 may be configured to record an epoch time signifying a beginning of capturing the audio content and thus beginning of the audio stream. The epoch time may include a month, a day, and a year. The time of day may include an hour, a minute, a second, a millisecond, a microsecond, and/or other time increments for precision.

The individual inputs may include timing information and/or other information. The timing information for the individual inputs may include timestamps. Timestamps may include a date, a time of day, and a time zone. The date may include a month, a day, and a year. The time of day may include an hour, a minute, a second, a millisecond, a microsecond, and/or other time increments for precision. The timestamp may be expressed in one of various formats including international date format, military time, 12-hour time, and/or other formats. The timing information for the spoken inputs may provide relative timing of multiple spoken inputs. The timing information for the spoken inputs may be determined based on the epoch time. The timing information for the spoken inputs may be individual to each spoken word. That is, each spoken word may include individual timing information. In some implementations, a first word utter of a spoken phrase may include the individual timing information in lieu of each of the individual spoken words. The timing information for the individual physical inputs may provide relative time of multiple physical inputs. The timing information for the inputs may provide relative time of multiple inputs (i.e., both physical inputs and spoken inputs).

Information processing component 112 may be configured to process the output signals of the physical user interface 108 to generate a physical input stream representing the individual physical inputs of the user to the physical user interface 108 as a function of time. The physical input stream may represent the physical manipulation to convey the individual physical inputs. The term “physical input stream” as used herein is for illustrative purposes only to indicate the temporal nature of a record of the physical inputs described. The term does not necessarily imply the record includes any and all physical manipulations by the user to the physical user interface 108 (e.g., may exclude those not associated with an input and/or command), and/or does not necessarily imply the entirety of the record is stored within a single file data object, or other logical information storage construct. The physical input stream may convey the individual physical inputs by the user through the physical interface 108 and the timing information for the individual physical inputs so that the physical inputs conveyed are in chronological order. In some implementations, the individual physical inputs may be associated with an individual command. For example, a touch-and-hold of a part of the touchscreen of the physical user interface 108 may be associated with a command to replace a value. Thus, in some implementations, the physical input stream may specify the commands based on association with the individual physical inputs. In some implementations, combinations of two or more physical inputs may specify individual commands. That is, two or more of the physical inputs in combination with the spoken inputs or not in combinations with the spoken inputs may specify individual commands. For example, a screen drag followed by a screen tap may specify a particular command.

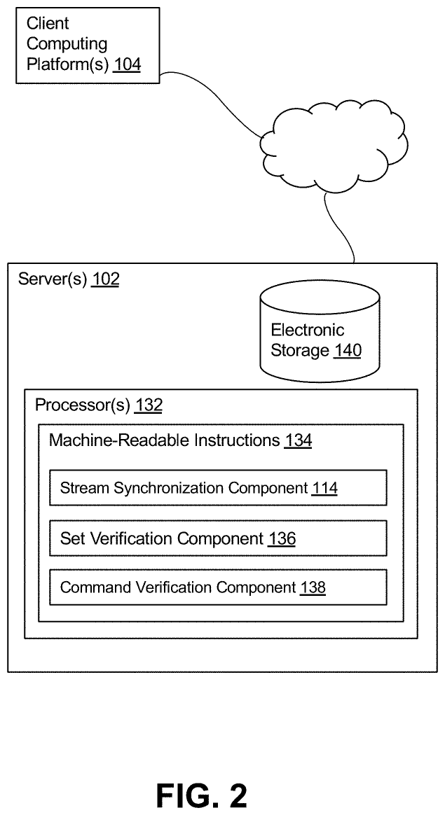

In some implementations, the individual physical inputs may specify locations. The locations may refer to file locations, locations or portions within a document displayed via the display of client computing platform 104, and/or other locations. For example, upon the user tapping the touchscreen with the stylus at a location representing User Comments within the document displayed, the spoken inputs may contribute to execution of a particular command. Information processing component 112 may be configured to transmit the physical input stream to server(s) 102 (i.e., stream synchronization component 114) in FIG. 2 via a network.

Information processing component 112 may be configured to process the captured audio information to generate an audio stream as a function of time. The audio stream may represent the spoken inputs uttered by the user and the timing information of the spoken inputs. The term “audio stream” as used herein is for illustrative purposes only to indicate the temporal nature of a record of the spoken inputs described. The term does not necessarily imply the record includes any and all audio content spoken by the user and captured by audio input sensor 110 (e.g., may exclude utterances by the user not associated with an input and/or command), and/or does not necessarily imply the entirety of the record is stored within a single file data object, or other logical information storage construct, nor implies an obtained audio for presentation such as music. In some implementations, the audio stream may include verbatim transcription of all utterances by the user that define the spoken inputs. In some implementations, the verbatim transcription may be generated from the audio stream in real-time or near real-time by external resources 126. The verbatim transcription may be an alternative to particular phrases or combinations of words that are determined to be relevant to the individual commands. In some implementations, the verbatim transcription may be used alternatively to interpret the commands (by command processing component 116). Information processing component 112 may be configured to transmit the audio stream to server(s) 102 (i.e., stream synchronization component 114) in FIG. 2 via the network.

FIG. 2 may illustrate an implementation of the system configured to facilitate real-time editing by voice based on individual inputs from a user and timing information for the individual inputs, in accordance with one or more implementations. FIG. 2 may illustrate server(s) 102 that may communicate with client computing platform(s) 104 of FIG. 1 via the network. Server(s) 102 may be configured by machine-readable instructions 134. Machine-readable instructions 134 may include one or more instruction components. The instruction components may include computer program components. The instruction components may include one or more of a stream synchronization component 114, a set verification component 136, a command verification component 138, and/or other instruction components.

Stream synchronization component 114 may be configured to receive the physical input stream, the audio stream, the epoch time, the timing information. and/or other information from client computing platform 104 of FIG. 1. Stream synchronization component 114 may be configured to synchronize the physical input stream and the audio stream based on the timing information for the individual input and the epoch time to convey relative timing between the inputs to client computing platform 104. The relative timing between the inputs may include the inputs to client computing platform 104 both (i) in the form of manipulations of the physical user interface 108 (i.e., the physical inputs) and (ii) in the form of spoken inputs included in the captured audio information (i.e., the spoken inputs). Synchronizing the physical input stream and the audio stream may refer to combining the timing information of the physical inputs with the timing information of the spoken inputs to convey the timing information of the physical inputs relative to the timing information of the spoken inputs and vice versa. Thus, the inputs, both the physical inputs and the spoken inputs, may occur sequentially in the synchronization and therefore the synchronization may include multiple commands. For example, a tap to a part of the touchscreen may occur, subsequently the user may utter “John has a tender stomach”, subsequently a tap to another part of the touchscreen may occur, followed by the user uttering “ultrasound of abdomen needed”. The physical input stream and the audio stream may be synchronized in an ongoing manner, that is so long as the physical inputs are being generated and the spoken inputs are being captured. The term “ongoing” as used herein may refer to continuing to perform an action (e.g., determine) periodically (e.g., every 30 seconds, every minute, every hour, etc.) until receipt of an indication to terminate. The indication to terminate may include powering off client computing platform 104, charging one or more of a battery of client computing platform 104, a particular physical input (e.g., pressing a button, selecting a virtual button), and/or other indications of termination. The synchronization may be completed upon indication of termination of generation of the physical inputs and/or indication of termination to capturing of the spoken inputs.

Stream synchronization component 114 may be configured to store the synchronized physical input stream and audio stream to electronic storage 140, to a cloud-based storage, and/or other storage. In some implementations, the synchronized physical input stream and audio stream may be stored in associated with a patient name, a date, a medical record number (MRN) of the patient, and/or other information. In some implementations, the date associated with the synchronized physical input stream and audio stream may be a timestamp of an initial input generated or captured. The synchronized physical input stream and audio stream may be retrieved by client computing platform 104 of FIG. 1 from electronic storage 140 for subsequent review by the user, a reviewer, and/or other healthcare personnel; transcription by a transcriber (e.g., a person or software); deletion by the user or other healthcare personnel; and/or other actions. In some implementations, stream synchronization component 114 may be configured to transmit the synchronized physical input stream and audio stream to client computing platform 104 and/or other servers via the network for further determinations and/or for storage.

Referring back to FIG. 1, command processing component 116 may be configured to receive the synchronized physical input stream and audio stream and/or other information from server(s) 102 (i.e., stream synchronization component 114) of FIG. 2 via the network. The synchronized physical input stream and audio stream and/or other information received may be stored in electronic storage 128 and/or other storage.

Command processing component 116 may be configured to determine, from the synchronized physical input stream and audio stream, sets of inputs that correspond to different individual commands. The sets of inputs may be determined from the ongoing physical input stream and the audio stream and the synchronization thereof. The determination of the sets of input may be performed in real-time or near real-time to determine one or more actions to execute as commands. In some implementations, determining the sets of inputs may include determining the commands from the individual physical inputs. In some implementations, determining the sets of inputs may include determining the commands from the verbatim transcription.

In some implementations, determining the sets of inputs may include command processing component 116 interpreting the commands from the spoken phrase to determine an action to execute. Interpreting the commands may include performing speech recognition on the spoken phrases and/or other spoken inputs and analyzing the recognized spoken inputs. In some implementations, the speech recognition may be performed by known techniques to determine text from the spoken phrases and/or the spoken inputs. Upon analyzing the recognized speech or the determined text, the action to execute may be determined. By way of non-limiting illustration, the actions may include inserting text, deleting text, modifying text, updating text, moving files (e.g., assessments, reports, images), deleting files, and/or other actions. In some implementations, particular words and/or phrases included in the determined text may correspond to one of the pre-set words and/or phrases that are relevant to particular commands. For example, a word “enter” may be recognized and correspond to words “insert” and “input” of which are relevant and correspond to a text insertion command.

As such, a first set of inputs may be determined corresponding to a first command, a second set of inputs may be determined corresponding to a second command, and/or other sets of inputs corresponding to other commands. The second set of inputs may be separate and discrete from the first set of inputs. The first set of inputs may include a first input in the form of a manipulation of the physical user interface 108 and a second input that is a spoken input. The second set of inputs may include a third input in the form of a manipulation of the physical user interface 108 and fourth input that is a spoken input.

Command processing component 116 may be configured to execute the commands corresponding to the sets of inputs. Executing the commands may include performing the actions determined. As such, the first command, the second command, and other commands may be executed. In some implementations, the actions determined and executed as the commands may be verified and, in some instances, reversed based on the verification. The verification may be performed by server(s) 102 and/or other servers.

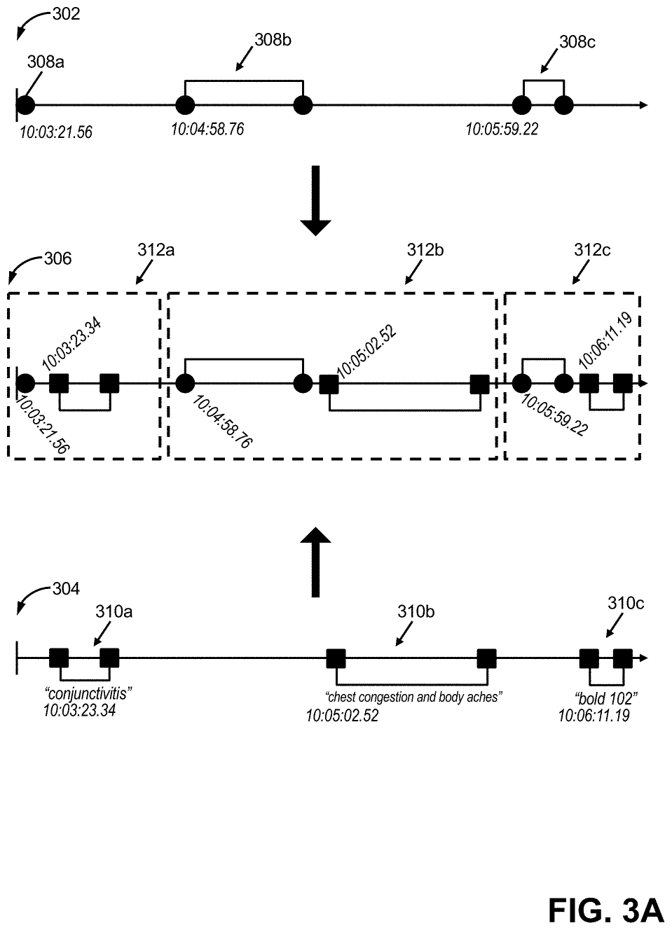

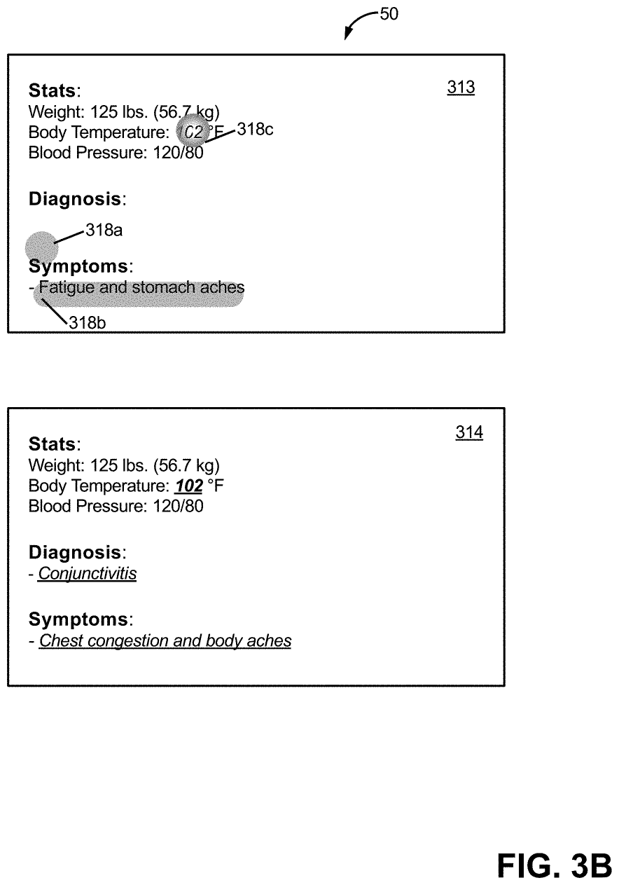

FIG. 3A-B may illustrate the system configured to facilitate real-time editing by voice based on individual inputs from a user and timing information for the individual inputs, in accordance with one or more implementations. FIG. 3A may illustrate a physical input stream 302, an audio stream 304, and a synchronized stream 306. Physical input stream 302 may include physical inputs 308a, 308b, and 308c by a user (e.g., a doctor). FIG. 3B may illustrate a touchscreen 50 (i.e., physical user interface) displaying a document 313. Physical input 308a may be a tap 318a to touchscreen 50 at time 10:03:21.56. Physical input 308b may be a screen drag 318b of touchscreen 50 at time 10:04:58.76. Physical input 308c may be a press-and-hold 318c of touchscreen 50 at time 10:05:59.22.

Referring to FIG. 3A, audio stream 304 may include spoken inputs 310a, 310b, and 310c. Spoken input 310a may include “conjunctivitis” spoken beginning at time 10:03:23.34. Spoken input 310b may include “chest congestion and body aches” beginning at time 10:05:02.52. Spoken input 310c may include “bold 102” beginning at time 10:06:11.19. Synchronized stream 306 may be a synchronization of physical input stream 302 and audio stream 304 where the timing of physical inputs 308a-c and spoken inputs 310a-c are relative to each other. Thus, chronologically physical input 308a occurred first followed by spoken input 310a, then physical input 308b, then spoken input 310b, then physical input 308c, and lastly spoken input 310c. Sets 312a, 312b, and 312c may be determined from synchronized stream 306 where set 312a corresponds to a first command, set 312b corresponds to a second command, and set 312c corresponds to a third command. That is, physical input 308a and spoken input 310a may correspond to the first command such as inserting “conjunctivitis” in a document 314 as a Diagnosis in FIG. 3B. Physical input 308b and spoken input 310b may correspond to the second command such as replacing existing text (i.e., “Fatigue and stomach aches” from screen drag 318b) with “chest congestion and body aches” as Symptoms in document 314. Physical input 308c and spoken input 310c may correspond to the third command such as bolding “102” in document 314.

Referring back to FIG. 2, in some implementations, set verification component 136 may be configured to perform at least the functions of command processing component 116 of FIG. 1 to determine verification sets of inputs that correspond to different individual commands or the same individual commands from the synchronized physical input stream and audio stream. Set verification component 136 may be verifying the sets of inputs determined by command processing component 116 by determining the verification sets of inputs.

Set verification component 136 may be configured to compare the verification sets of inputs with the sets of inputs determined by command processing component 116. In some implementations, the verification sets of inputs may differ than the sets of inputs determined by command processing component 116 based on the comparison. Thus, in some implementations, the verification sets of inputs may correspond to different individual commands than executed by command processing component 116. In some implementations, the verification sets of inputs may correspond to the same individual commands than determined and executed by command processing component 116. In such instances, correcting the command executed may not be necessary.

In some implementations, upon the verification sets of inputs differing from the sets of inputs determined by command processing component 116, command verification component 138 may be configured to determine a correction command. The correction command may be executed subsequent to the command originally executed in order to correct the command originally executed. The correction command may include one or more of the actions to accurately accomplish the command desired by the user responsive to the command already executed in attempts to accomplish to the command desired by the user. Command verification component 138 may be configured to transmit the correction command to client computing platform(s) 104 for execution by command processing component 116. In some implementations, command verification component 138 may be configured to execute the correction command.

In some implementations, upon the verification sets of inputs differing from the sets of inputs determined by command processing component 116, command verification component 138 may be configured to determine correction instructions. The correction instructions may include instructions to reverse the commands executed by command processing component 116 and either re-execute the commands executed by command processing component 116 or execute the different commands that correspond to the verification sets of inputs, and/or other actions. In some implementations, the re-execution of the commands may perform a different action than performed by command processing component 116 with the same intent as the original command. In some implementations, executing the different commands may perform a different action than performed by command processing component 116.

In some implementations, the correction instructions may be executed by command verification component 138. In some implementations, the correction instructions may be transmitted to the client computing platform(s) 104 for execution by command processing component 116. By sets of inputs being determined by two entities, client computing platform(s) 104 and server(s) 102, and compared the actions performed as executed commands may be more accurate.

As such, referring to FIG. 1, in some implementations, command processing component 116 may be configured to receive the correction command. Subsequently, command processing component 116 may be configured to execute the correction command. For example, text may have been mistakenly added under a first section of a document during execution of the command originally. The correction command executed may move such added text from the first section to a second section, where the second section may be distinct from the first section.

In some implementations, command processing component 116 may be configured to receive the correction instructions, the verification sets of inputs that correspond to the different individual commands, and/or other information. Subsequently, command processing component 116 may be configured to execute the correction instructions. For example, the command originally executed that added the text to the first section may be reversed so that the adding of the text to the first section is undone. Subsequently, the different command may be executed so that the text is added under the second section.

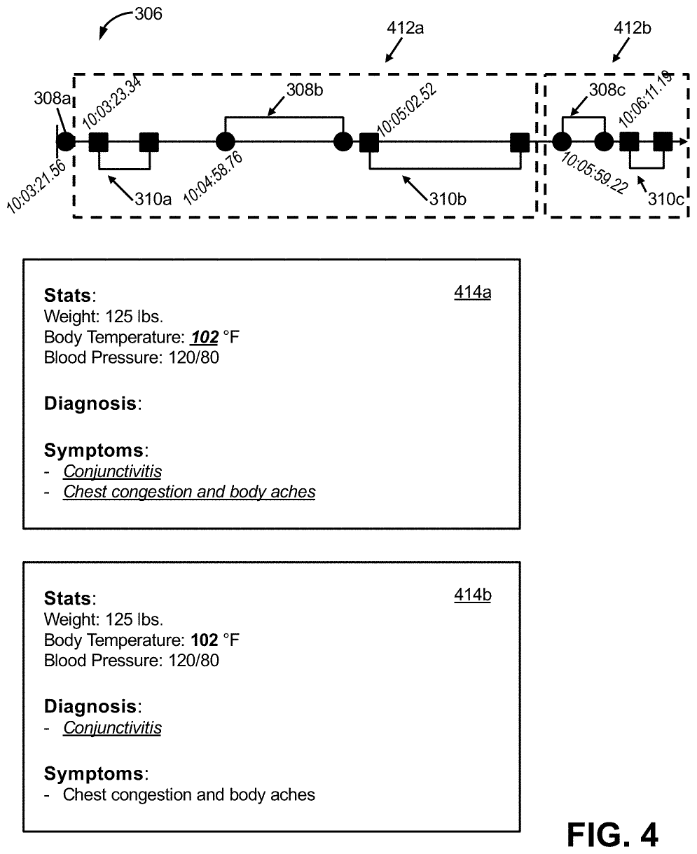

FIG. 4 may illustrate the system configured to facilitate real-time editing by voice based on individual inputs from a user and timing information for the individual inputs, in accordance with one or more implementations. FIG. 4 may illustrate synchronized stream 306 from FIG. 3A-B that is based on tap 318a as physical input 308a, screen drag 318b as physical input 308b, and press-and-hold 318c as physical input 308c and spoken inputs 310a-c. Sets 412a and 412b may be determined where set 412a corresponds to a first command and set 412b corresponds to a second command. That is, spoken input 310a, physical input 308b, and spoken input 310b (i.e., set 412a) may correspond to the first command such as inserting “conjunctivitis” and replacing existing text (dragged over during the screen drag) with “chest congestion and body aches” under Symptoms in a document 414a. Physical input 308c and spoken input 310c (i.e., set 412b) may correspond to the second command such as bolding “102” in document 414a. Upon a server (e.g., server(s) 102 of FIG. 2) obtaining synchronized stream 306, the server may verify the sets of inputs determined (e.g., set verification component 136 of FIG. 2). The server may determine three sets of inputs that correspond to three individual commands as illustrated in FIG. 3A as sets 312a-c. Thus, the server (e.g., command verification component 138) may execute a correction command so that “conjunctivitis” is under Diagnosis in a corrected document 414b (i.e., the same as document 314 in FIG. 3B).

FIG. 5 may illustrate the system configured to facilitate real-time editing by voice based on individual inputs from a user and timing information for the individual inputs, in accordance with one or more implementations. FIG. 5 may illustrate continued physical input stream 302, audio stream 304, and synchronized stream 306 from FIG. 3A. Physical input stream 302 may further include physical inputs 308d and 308e by the user. Touchscreen 50 (the same as FIG. 3B) may display corrected document 414b (from FIG. 4). Physical input 308d may be a screen drag 318d of touchscreen 50 at time 10:08:25.10. The user may decide to abandon their desired command and thus utter spoken input 310d (e.g., “never mind”) at time 10:09:10.96 as conveyed in audio stream 304. As such, synchronized stream 306 may include physical input 308d and spoken input 310d, chronologically. Set 312d may be determined from synchronized stream 306 where set 312d corresponds to physical input 308d instigating a command and spoken input 310d terminating such command.

The user may perform screen drag 318d again at time 10:10:51.17 by which physical input 308e is generated and included in physical input stream 302. The user may decide, again, to abandon their desired command and thus select a button (not pictured) at time 10:11:01.00 by which physical input 308f is generated and included in physical input stream 302. Synchronized stream 306 may further include physical input 308e and physical input 308f, chronologically after spoken input 310d. Set 312e may be determined from synchronized stream 306 where set 312e corresponds to physical input 308e instigating another command and physical 308f terminating such command.

Referring back to FIG. 1, in some implementations, server(s) 102, client computing platform(s) 104, and/or external resources 126 may be operatively linked via one or more electronic communication links. For example, such electronic communication links may be established, at least in part, via the network such as the Internet and/or other networks. It will be appreciated that this is not intended to be limiting, and that the scope of this disclosure includes implementations in which server(s) 102, client computing platform(s) 104, and/or external resources 126 may be operatively linked via some other communication media.

A given client computing platform 104 may include one or more processors 130 configured to execute computer program components, electronic storage 128, and/or other components. The computer program components may be configured to enable an expert or user associated with the given client computing platform 104 to interface with system 100 and/or external resources 126, and/or provide other functionality attributed herein to client computing platform(s) 104. By way of non-limiting example, the given client computing platform 104 may include one or more of a desktop computer, a laptop computer, a handheld computer, a tablet computing platform, a NetBook, a Smartphone, a gaming console, and/or other computing platforms.

Client computing platform(s) 104 may include communication lines, or ports to enable the exchange of information with a network and/or other computing platforms. Illustration of client computing platform(s) 104 in FIG. 1 is not intended to be limiting. Client computing platform(s) 104 may include a plurality of hardware, software, and/or firmware components operating together to provide the functionality attributed herein to client computing platform(s) 104. For example, client computing platform(s) 104 may be implemented by a cloud of computing platforms operating together as client computing platform(s) 104.

Electronic storage 128 may comprise non-transitory storage media that electronically stores information. The electronic storage media of electronic storage 128 may include one or both of system storage that is provided integrally (i.e., substantially non-removable) with client computing platform(s) 104 and/or removable storage that is removably connectable to client computing platform(s) 104 via, for example, a port (e.g., a USB port, a firewire port, etc.) or a drive (e.g., a disk drive, etc.). Electronic storage 128 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 128 may include one or more virtual storage resources (e.g., cloud storage, a virtual private network, and/or other virtual storage resources). Electronic storage 128 may store software algorithms, information determined by processor(s) 130, information received from server(s) 102 (of FIG. 2), information received from client computing platform(s) 104, and/or other information that enables client computing platform(s) 104 to function as described herein.

Processor(s) 130 may be configured to provide information processing capabilities in client computing platform(s) 104. As such, processor(s) 130 may include one or more of a digital processor, an analog processor, a digital circuit designed to process information, an analog circuit designed to process information, a state machine, and/or other mechanisms for electronically processing information. Although processor(s) 130 is shown in FIG. 1 as a single entity, this is for illustrative purposes only. In some implementations, processor(s) 130 may include a plurality of processing units. These processing units may be physically located within the same device, or processor(s) 130 may represent processing functionality of a plurality of devices operating in coordination. Processor(s) 130 may be configured to execute components 112 and/or 116, and/or other components. Processor(s) 130 may be configured to execute components 112 and/or 116, and/or other components by software; hardware; firmware; some combination of software, hardware, and/or firmware; and/or other mechanisms for configuring processing capabilities on processor(s) 130. As used herein, the term “component” may refer to any component or set of components that perform the functionality attributed to the component. This may include one or more physical processors during execution of processor readable instructions, the processor readable instructions, circuitry, hardware, storage media, or any other components.

It should be appreciated that although components 112 and/or 116 are illustrated in FIG. 1 as being implemented within a single processing unit, in implementations in which processor(s) 130 includes multiple processing units, one or more of components 112 and/or 116 may be implemented remotely from the other components. The description of the functionality provided by the different components 112 and/or 116 described below is for illustrative purposes, and is not intended to be limiting, as any of components 112 and/or 116 may provide more or less functionality than is described. For example, one or more of components 112 and/or 116 may be eliminated, and some or all of its functionality may be provided by other ones of components 112 and/or 116. As another example, processor(s) 130 may be configured to execute one or more additional components that may perform some or all of the functionality attributed below to one of components 112 and/or 116.

Server(s) 102 may include electronic storage 140, one or more processors 132, and/or other components. Server(s) 102 may include communication lines, or ports to enable the exchange of information with a network and/or other computing platforms. Illustration of server(s) 102 in FIG. 2 is not intended to be limiting. Server(s) 102 may include a plurality of hardware, software, and/or firmware components operating together to provide the functionality attributed herein to server(s) 102. For example, server(s) 102 may be implemented by a cloud of computing platforms operating together as server(s) 102.

Electronic storage 140 may comprise non-transitory storage media that electronically stores information. The electronic storage media of electronic storage 140 may include one or both of system storage that is provided integrally (i.e., substantially non-removable) with server(s) 102 and/or removable storage that is removably connectable to server(s) 102 via, for example, a port (e.g., a USB port, a firewire port, etc.) or a drive (e.g., a disk drive, etc.). Electronic storage 128 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 140 may include one or more virtual storage resources (e.g., cloud storage, a virtual private network, and/or other virtual storage resources). Electronic storage 140 may store software algorithms, information determined by processor(s) 132, information received from server(s) 102, information received from client computing platform(s) 104, and/or other information that enables server(s) 102 to function as described herein.

Processor(s) 132 may be configured to provide information processing capabilities in server(s) 102. As such, processor(s) 132 may include one or more of a digital processor, an analog processor, a digital circuit designed to process information, an analog circuit designed to process information, a state machine, and/or other mechanisms for electronically processing information. Although processor(s) 132 is shown in FIG. 2 as a single entity, this is for illustrative purposes only. In some implementations, processor(s) 132 may include a plurality of processing units. These processing units may be physically located within the same device, or processor(s) 132 may represent processing functionality of a plurality of devices operating in coordination. Processor(s) 132 may be configured to execute components 114, 136, and/or 138, and/or other components. Processor(s) 132 may be configured to execute components 114, 136, and/or 138, and/or other components by software; hardware; firmware; some combination of software, hardware, and/or firmware; and/or other mechanisms for configuring processing capabilities on processor(s) 132.

It should be appreciated that although components 114, 136, and/or 138 are illustrated in FIG. 2 as being implemented within a single processing unit, in implementations in which processor(s) 132 includes multiple processing units, one or more of components 114, 136, and/or 138 may be implemented remotely from the other components. The description of the functionality provided by the different components 114, 136, and/or 138 described below is for illustrative purposes, and is not intended to be limiting, as any of components 114, 136, and/or 138 may provide more or less functionality than is described. For example, one or more of components 114, 136, and/or 138 may be eliminated, and some or all of its functionality may be provided by other ones of components 114, 136, and/or 138. As another example, processor(s) 132 may be configured to execute one or more additional components that may perform some or all of the functionality attributed below to one of components 114, 136, and/or 138. It is to be understood that some or all functionality provided by components 112, 114, 116, 136, and/or 138 of FIG. 1-2 may be performed by client computing platform 104 and/or server(s) 102 and FIG. 1-2 are for illustrative purposes only.

External resources 126 may include sources of information outside of system 100, external entities participating with system 100, and/or other resources. In some implementations, some or all of the functionality attributed herein to external resources 126 may be provided by resources included in system 100.

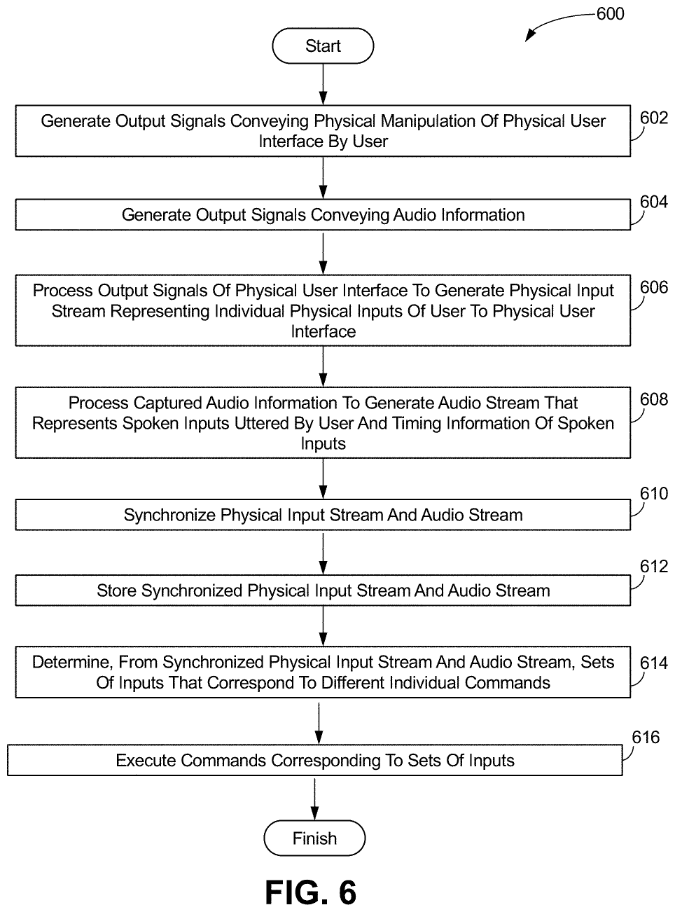

FIG. 6 illustrates a method 600 to facilitate real-time editing by voice based on individual inputs from a user and timing information for the individual inputs, in accordance with one or more implementations. The operations of method 600 presented below are intended to be illustrative. In some implementations, method 600 may be accomplished with one or more additional operations not described, and/or without one or more of the operations discussed. Additionally, the order in which the operations of method 600 are illustrated in FIG. 6 and described below is not intended to be limiting.

In some implementations, method 600 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 600 in response to instructions stored electronically on an electronic storage medium. The one or more processing devices may include one or more devices configured through hardware, firmware, and/or software to be specifically designed for execution of one or more of the operations of method 600.

An operation 602 may include generating output signals conveying physical manipulation of the physical user interface by a user. The output signals may be generated by a physical user interface associated with a client computing platform. The user may be enabled to generate physical inputs to the client computing platform through the physical manipulation of the physical user interface. Individual inputs may be associated with commands for the client computing platform. Operation 602 may be performed by physical user interface 108, in accordance with one or more implementations.

An operation 604 may include generating output signals conveying audio information. The output signals may be generated by an audio input sensor associated with the client computing platform. The audio information may define audio content captured by the audio input sensor. The audio content may include spoken inputs uttered by the user. The inputs may include the physical inputs and the spoken inputs. Operation 604 may be performed by audio input sensor 110, in accordance with one or more implementations.

An operation 606 may include processing the output signals of the physical user interface to generate a physical input stream representing the individual physical inputs of the user to the physical user interface. The physical input stream may convey the individual physical inputs by the user through the physical interface and timing information for the individual physical inputs. Operation 606 may be performed by one or more hardware processors configured by machine-readable instructions including a component that is the same as or similar to information processing component 112, in accordance with one or more implementations.

An operation 608 may include processing the captured audio information to generate an audio stream that represents the spoken inputs uttered by the user and timing information of the spoken inputs. Operation 608 may be performed by one or more hardware processors configured by machine-readable instructions including a component that is the same as or similar to information processing component 112, in accordance with one or more implementations.

An operation 610 may include synchronizing the physical input stream and the audio stream to convey relative timing between inputs to the client computing platform including inputs to the client computing platform both in the form of manipulations of the physical user interface and in the form of spoken inputs included in the captured audio information. Operation 610 may be performed by one or more hardware processors configured by machine-readable instructions including a component that is the same as or similar to stream synchronization component 114, in accordance with one or more implementations.

An operation 612 may include storing the synchronized physical input stream and audio stream. Operation 612 may be performed by one or more hardware processors configured by machine-readable instructions including a component that is the same as or similar to stream synchronization component 114, in accordance with one or more implementations.

An operation 614 may include determining, from the synchronized physical input stream and audio stream, sets of inputs that correspond to different individual commands. As such, a first set of inputs may be determined corresponding to a first command, and a second set of inputs may be determined corresponding to a second command. The second set of inputs may be separate and discrete from the first set of inputs. Operation 614 may be performed by one or more hardware processors configured by machine-readable instructions including a component that is the same as or similar to command processing component 116, in accordance with one or more implementations.

An operation 616 may include executing the commands corresponding to the sets of inputs. As such, the first command and the second command may be executed. Operation 616 may be performed by one or more hardware processors configured by machine-readable instructions including a component that is the same as or similar to command processing component 116, in accordance with one or more implementations.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

1. A system configured to facilitate real-time editing by voice based on individual inputs from a user and timing information for the individual inputs, the system comprising:
a physical user interface associated with a client computing platform, the physical user interface being configured to generate output signals conveying physical manipulation of the physical user interface by a user, wherein the user is enabled to generate physical inputs to the client computing platform through the physical manipulation of the physical user interface, and wherein individual inputs are associated with commands for the client computing platform;
an audio input sensor associated with the client computing platform, the audio input sensor being configured to generate output signals conveying audio information, wherein the audio information defines audio content captured by the audio input sensor, the audio content including spoken inputs uttered by the user, wherein the inputs include the physical inputs and the spoken inputs; and
one or more processors configured by machine-readable instructions to:
process the output signals of the physical user interface to generate a physical input stream representing the individual physical inputs of the user to the physical user interface, wherein the physical input stream conveys the individual physical inputs consecutively performed by the user through the physical interface and timing information for the individual physical inputs, wherein the physical inputs are performed in relation to a document displayed via a screen of the client computing platform;
process the captured audio information to generate an audio stream that represents the spoken inputs uttered by the user and timing information of the spoken inputs;
synchronize the physical input stream and the audio stream to convey relative timing between inputs to the client computing platform including inputs to the client computing platform both (i) in the form of manipulations of the physical user interface and (ii) in the form of spoken inputs included in the captured audio information to generate a synchronized physical input stream and audio stream that conveys both the physical inputs and the spoken inputs occurring in a temporal sequence;
store the synchronized physical input stream and audio stream;
determine, from the synchronized physical input stream and audio stream, sets of inputs that correspond to different individual commands such that a first set of inputs is determined corresponding to a first command, and a second set of inputs, separate and discrete from the first set of inputs, is determined corresponding to a second command, wherein:
the first set of inputs includes a first input in the form of a manipulation of the physical user interface and a second input that is a spoken input, and
the second set of inputs includes a third input in the form of a manipulation of the physical user interface and fourth input that is a spoken input;
interpret the individual commands from the spoken inputs and the physical inputs included in the sets of inputs to determine actions to execute; and
execute, based on the interpretation, the commands corresponding to the sets of inputs by executing the actions determined such that the first command and the second command are executed.

2. The system of claim 1, wherein the physical input stream represents the physical manipulation to convey the individual physical inputs.

3. (canceled)

4. The system of claim 1, wherein the timing information for the individual inputs includes timestamps.

5. The system of claim 4, wherein the timing information for the individual physical inputs provides relative time of multiple physical inputs.

6. The system of claim 4, wherein the timing information for the spoken inputs provides relative timing of multiple spoken inputs.

7. The system of claim 1, wherein the audio stream includes spoken phrases by the user.

8. The system of claim 1,
wherein interpreting the commands includes performing speech recognition on the spoken inputs and analyzing the recognized speech.

9. The system of claim 1, wherein the one or more processors are further configured by the machine-readable instructions to transmit the synchronized physical input stream and audio stream to a server.

10. The system of claim 1, wherein the commands include editing a text presented via a display of the client computing platform.

11. A method to facilitate real-time editing by voice based on individual inputs from a user and timing information for the individual inputs, the method comprising:
generating output signals, by a physical user interface associated with a client computing platform, conveying physical manipulation of the physical user interface by a user, wherein the user is enabled to generate physical inputs to the client computing platform through the physical manipulation of the physical user interface, and wherein individual inputs are associated with commands for the client computing platform;
generating output signals, by an audio input sensor associated with the client computing platform, conveying audio information, wherein the audio information defines audio content captured by the audio input sensor, the audio content including spoken inputs uttered by the user, wherein the inputs include the physical inputs and the spoken inputs;
processing the output signals of the physical user interface to generate a physical input stream representing the individual physical inputs of the user to the physical user interface, wherein the physical input stream conveys the individual physical inputs consecutively performed by the user through the physical interface and timing information for the individual physical inputs, wherein the physical inputs are performed in relation to a document displayed via a screen of the client computing platform;
processing the captured audio information to generate an audio stream that represents the spoken inputs uttered by the user and timing information of the spoken inputs;
synchronizing the physical input stream and the audio stream to convey relative timing between inputs to the client computing platform including inputs to the client computing platform both (i) in the form of manipulations of the physical user interface and (ii) in the form of spoken inputs included in the captured audio information to generate a synchronized physical input stream and audio stream that conveys both the physical inputs and the spoken inputs occurring in a temporal sequence;
storing the synchronized physical input stream and audio stream;
determining, from the synchronized physical input stream and audio stream, sets of inputs that correspond to different individual commands such that a first set of inputs is determined corresponding to a first command, and a second set of inputs, separate and discrete from the first set of inputs, is determined corresponding to a second command, wherein:
the first set of inputs includes a first input in the form of a manipulation of the physical user interface and a second input that is a spoken input, and
the second set of inputs includes a third input in the form of a manipulation of the physical user interface and fourth input that is a spoken input;
interpreting the individual commands from the spoken inputs and the physical inputs included in the sets of inputs to determine actions to execute; and
executing, based on the interpretation, the commands corresponding to the sets of inputs by executing the actions determined such that the first command and the second command are executed.

12. The method of claim 11, wherein the physical input stream represents the physical manipulation to convey the individual physical inputs.

13. (canceled)

14. The method of claim 11, wherein the timing information for the individual inputs includes timestamps.

15. The method of claim 14, wherein the timing information for the individual physical inputs provides relative time of multiple physical inputs.

16. The method of claim 14, wherein the timing information for the spoken inputs provides relative timing of multiple spoken inputs.

17. The method of claim 11, wherein the audio stream includes spoken phrases by the user.

18. The method of claim 11,
wherein interpreting the commands includes performing speech recognition on the spoken inputs and analyzing the recognized speech.

19. The method of claim 11, wherein the one or more processors are further configured by the machine-readable instructions to transmit the synchronized physical input stream and audio stream to a server.

20. The method of claim 11, wherein the commands include editing a text presented via a display of the client computing platform.
